(19) United States
(12) Patent Application Publication
(10) Pub. No.: US 2004/0209368 A1
(43) Pub. Date: Oct. 21, 2004

(54) Process for producing sporidiobolus ruineniae strains with improved coenzyme Q10 production

(75) Inventors: Rupert Pfaller, Munchen (DE); Susanne Leonhartsberger, Munchen (DE)

Correspondence Address: COLLARD & ROE, P.C., 1077 Northern Boulevard, Roslyn, NY, 11576, US

(73) Assignee: Consortium Fur Electrochemische Industrie GmbH

(21) Appl. No.: 10/803,841

(22) Filed: Mar. 18, 2004

(30) Foreign Application Priority Data
Apr. 17, 2003 (DE) 103 17 877.5

(51) Int. Cl. C12N 9/88; C12N 15/74; C12N 1/21

(52) U.S. Cl. 435/471; 435/252.3

(57) ABSTRACT

A process for producing a mutagenized Sporidiobolus ruineniae strain having a Q10 productivity of greater than 1.38 mg of Q10/g of dry biomass, wherein

(a) a Sporidiobolus ruineniae strain is subjected to genetic manipulation by mutagenesis, and

(b) the mutagenized Sporidiobolus ruineniae strain is subjected to a selection where the mutagenized Sporidiobolus ruineniae strain is cultivated under conditions which inhibit growth of the Sporidiobolus ruineniae strain employed in (a), the conditions being chosen so that the mutagenized strain overcomes the growth inhibition through a Q10 production which is increased by comparison with the Sporidiobolus ruineniae strain employed in (a), grows in a fermentation medium and

(c) is isolated from the fermentation medium.

### BACKGROUND OF THE INVENTION

1. Field of the Invention

The invention relates to a process for producing Sporidiobolus ruineniae strains with improved coenzyme Q10 production, to strains of the species Sporidiobolus ruineniae (S. ruineniae) obtainable in this way, and to the use of the strains for the production of ubiquinone 10 (coenzyme Q10, called Q10 hereinafter).

2. The Prior Art

Coenzyme Q10 is a compound from the family of ubiquinones having the general formula:

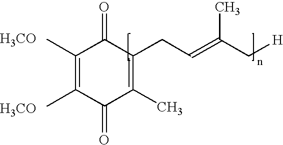

The chemical structure of Q10 is described by the formula given above from a ring-substituted quinone derivative and a hydrophobic chain of n isoprene units of the general formula —CH₂—CH═C(CH₃)—CH₂—. The number n of isoprene units is specific for the ubiquinone of a given organism.

Q10 is a ubiquinone of the general formula given above, where the number n of isoprene units in Q10 amounts to n=10 (corresponding to an isoprene chain of 50 C atoms). Q10 occurs widely in natural organisms, especially in humans, other higher organisms from the animal and plant kingdoms, but also in various species of bacteria, fungi or yeasts. Other known ubiquinones are Q6 (baker's yeast), Q8 (E. coli), Q9 (Rhodobacter capsulatus).

The chemical structure of Q10 determines its physiological/chemical function and the localization within a cell. The quinoid structure possesses the biochemical effector function and enables reversible electron uptake (reduction by two electrons, hydroquinone form) and release (oxidation, quinone form). An intermediate stage also known is the semiquinone form (uptake or release of only one electron). Localization of Q10 in cell membranes is the effect of the hydrophobic isoprene chain.

Known biochemical and physiological functions of Q10 include (summarized in the review article “Biochemical Functions of Coenzyme Q10”. F. L. Crane, J. of the Am. College of Nutrition (2001) 20: 591-598):

electron carrier in the respiratory chain (energy conversion).

protection from oxidative damage in cell membranes (antioxidant function and regeneration of other antioxidants).

stimulation of cell growth and inhibition of cell death.

Because of the biochemical and physiological function, Q10 is used in various sectors such as drugs, food products and cosmetics.

Known areas of use of Q10 in the pharmaceutical sector are heart failure and other cardiovascular diseases.

Q10 is employed in the food product sector as food supplement in vitamin preparations.

Q10 is now used in cosmetics as additive in skin creams, and the use in dental care compositions is also being tested.

No cost-effective chemical processes for synthesizing Q10 are known. Q10 is produced by extraction from naturally occurring sources. The Q10 content in naturally occurring sources is generally so low that obtaining Q10 by extraction is uneconomic. There are only a few natural sources in which the Q10 content is sufficiently high (e.g. a few microorganisms or vegetable oils). This is why, despite an increase in demand, the price of Q10 is still very high.

Various microorganisms with comparatively high Q10 production have been described. Bacterial producers include inter alia strains of the genera Agrobacterium, Rhodobacter or Hyphomonas. High Q10 productivity has been described in some cases, but commercial utilization is unknown.

Known yeast strains with Q10 production are representatives for example of the genera Sporobolomyces, Rhodotorula, Oosporidium and Sporidiobolus. Specially, strains of the species Sporidiobolus ruineniae were recognized as good producers of Q10 as long ago as 1978 (U.S. Pat. No. 4,070,244), with a Q10 productivity of 0.92 mg/g of biomass, among the highest productivities of known Q10-producing yeast strains.

Despite the early discovery of the property of Sporidiobolus ruineniae of being a good Q10 producer, no improvements which could have led to commercial utilization of Sporidiobolus ruineniae for Q10 production have been reported since. Specifically, no processes for isolating S. ruineniae strains with improved Q10 production, and no further development of the fermentation process for Q10 production are known, suggesting that there are difficulties in the further development of Sporidiobolus ruineniae as producer system for Q10 production. These difficulties are overcome by the present invention.

### SUMMARY OF THE INVENTION

The invention relates to a process for producing a mutagenized Sporidiobolus ruineniae strain having a Q10 productivity of greater than 1.38 mg of Q10/g of dry biomass, wherein

a) a Sporidiobolus ruineniae strain is subjected to genetic manipulation by mutagenesis, and

b) the mutagenized Sporidiobolus ruineniae strain is subjected to a selection where the mutagenized Sporidiobolus ruineniae strain is cultivated under conditions which inhibit growth of the Sporidiobolus ruineniae strain employed in a), the conditions being chosen so that the mutagenized strain overcomes the growth inhibition through a Q10 production which is increased by comparison with the Sporidiobolus ruineniae strain employed in a), grows in a fermentation medium and

c) is isolated from the fermentation medium.

Two metabolic pathways are involved in the biosynthesis of Q10 in microorganisms, namely the synthetic pathway to aromatic amino acids and the isoprenoid metabolism. A detailed survey of the biosynthesis of ubiquinones is to be found in “Ubiquinone Biosynthesis in Microorganisms”. R. Meganathan, FEMS Microbiology Letters (2001) 203: 131-139.

Through knowledge of the biochemical function and of the biosynthetic pathway it is possible in an inventive manner to isolate strains with improved Q10 production. The principle of the process (screening) of the invention is based on selective cell growth: S. ruineniae strains are cultivated under conditions which normally suppress cell growth. The growth-inhibiting conditions are chosen so that the cells are able to overcome the growth inhibition through increased Q10 production, and are able to grow again. Increased Q10 production by the improved strain is to be observed subsequent to the screening even in the absence of the growth-inhibiting substance.

The increased Q10 production can be brought about in various ways, but is always based on genetic modification of the S. ruineniae strain. The genetic modification leading to increased Q10 production in S. ruineniae is preferably effected by genetic manipulations based on the action of mutagenic substances or high-energy radiation on the S. ruineniae genome (mutagenesis), by fusing different S. ruineniae cells and the genetic recombination (called “genome shuffling”) elicted thereby, or by targeted intervention in the Q10 biosynthetic pathway using genetic engineering methods. Mutants with altered properties such as, for example, increased Q10 production can be identified by suitable selection conditions.

Substances suitable for the mutagenesis are N-methyl-N′-nitro-N-nitrosoguanidine (NG), ethyl methanesulfonate (EMS), dimethyl sulfate (DMS), and all other comparable mutagenic substances. Genotoxic radiation is high-energy, short-wavelength UV light, x-rays or radioactive emission.

However, any other type of genotoxic substances or radiations are suitable for bringing about a random alteration in the genetic material with the effect of increasing Q10 production in a mutant of S. ruineniae.

NG, EMS and UV radiation is preferably used for the mutagenesis. NG and UV radiation is particularly preferably used for the mutagenesis.

The mutagen is employed for the mutagenesis in an amount preferably bringing about a kill rate of from 30 to 99% of the mutagenized S. ruineniae cells before the subsequent selection. The preferred kill rate within the mutagenesis is from 40 to 90% of the S. ruineniae cells. A kill rate of from 50 to 90% of the S. ruineniae cells is particularly preferred. This is achieved for example by an NG concentration of 0.01-0.06 mg/ml.

Various methods can be employed to isolate mutants with improved Q10 production by selective cell growth in the manner according to the invention.

Conditions of selective cell growth can be generated at the level of the Q10 biosynthetic pathway. The selection conditions inhibit according to the invention individual biosynthetic steps in the Q10 biosynthetic pathway. These include all steps of the biosynthetic pathway of aromatic amino acids to 4-hydroxybenzoic acid and finally to tyrosine and all steps of the biosynthetic pathway to the isoprene side chain.

Inhibition of the biosynthetic pathway of aromatic amino acids is preferably at the stage of shikimate to chorismate through a competitive inhibitor of this biochemical synthetic step such as N-phosphonomethylglycine (also known under the name glyphosate, or Round Up).

Competitive inhibition of the synthesis of the isoprene side chain at the level of the synthesis of mevalonic acid is also preferred. Competitive inhibitors of the enzyme HMG-CoA reductase (called statins or cholesterol-lowering agents) are particularly suitable. Suitable selective inhibitors are statins such as lovastatin, cerivastatin, atorvastatin and compactin (mevastatin), each in their free or their lactone form, and each type of inhibitors which acts through inhibition of HMG-CoA reductase, for example mevalonate, which influences the synthesis of HMG-CoA reductase (Dimster-Denk, 1994). Lovastatin and cerivastatin are particularly preferred.

Further reaction steps from the biosynthetic pathways leading to isoprenoids which are suitable for the selection of S. ruineniae strains with improved Q10 production are, but are not restricted to, the reaction steps which lead to geranyl pyrophosphate, to farnesyl pyrophosphate, to squalene or, for example, to phytoene.

Biosynthesis of the Q10 quinone framework includes three methylation steps, with S-adenosylmethionine acting as donor of the methyl group. Strains with an increased level of S-adenosylmethionine which are capable of increased Q10 production can be isolated for example using the selective agent ethionine, a methionine analog.

A further preferred process of the invention for selecting S. ruineniae strains with improved Q10 production takes place via the biochemical function of Q10. Based on the antioxidant effect of Q10, all conditions which generate oxidative stress are suitable for a selection process of the invention. Agents which generate oxidative stress are, for example, activated oxygen species. Among these are hydrogen peroxide, including all possible peroxides derived from hydrogen peroxide and all reactive oxidative species generated in situ from reaction of hydrogen peroxide with other substances. One example thereof is the combination of hydrogen peroxide and linolenic acid, or unsaturated fatty acids in general. The generation of reactive lipid peroxides from mono- or polyunsaturated fatty acids is described in Do et al., Proc. Natl. Acad. Sci. USA (1996) 93: 7534-7539. Among the reactive oxygen species are also the superoxide radical, and compounds such as paraquat (1,1′-dimethyl-4,4′-bipyridinium dichloride, also called methylviologen or gramoxone) which are suitable for generating superoxide radicals. Also belonging to this class are the hydroxyl radical and compounds suitable for forming hydroxyl radicals. It is additionally possible to exert oxidative stress on the cells by subjecting them to a heat shock.

Preferred agents for an oxidative stress selection process are hydrogen peroxide, peroxides derived therefrom, and reactive oxidative species generated from the combination of hydrogen peroxide with other substances, and compounds such as paraquat which are capable of forming the superoxide radical, as well as unsaturated fatty acids such as linolenic acid. Particularly preferred agents for an oxidative stress selection process are hydrogen peroxide, paraquat and linolenic acid, and other mono- or polyunsaturated fatty acids or a combination of these agents.

A second biochemical function of Q10 suitable as further preferred process of the invention for selecting S. ruineniae strains with improved Q10 production is the function of Q10 as electron carrier in the respiratory chain. Because of this function, inhibitors able to impair the function of the respiratory chain are suitable for selecting cells with improved Q10 production. Q10 acts in the respiratory chain as electron carrier between complexes I and II on the one hand (electron uptake) and complex III on the other hand (electron release). The function of Q10 in the respiratory chain is summarized in the review article “Biochemical Functions of Coenzyme Q10”. F. L. Crane, J. of the Am. College of Nutrition (2001) 20: 591-598.

Various compounds able as inhibitors to impair the function of the respiratory chain are known (see “Mitochondria” by A. Tzagoloff, pages 92-95, 1982 Plenum Press New York, ISBN 0-306-40778-7). Besides general inhibitors such as, for example, cyanide or carbon monoxide, particularly suitable for the selection process of the invention for isolating S. ruineniae strains with improved Q10 production are structural analogs of Q10. Examples of suitable inhibitors of the respiratory chain are compounds such as antimycins, specifically antimycin A, rotenone, amobarbital, capsaicin, MPP+, myxothiazole, mucidin, menadione or piericidin. Preferred inhibitors are menadione, rotenone, antimycin A and piericidin. Particularly preferred inhibitors are menadione, antimycin A and piericidin.

These selective agents for isolating S. ruineniae strains with improved Q10 production can be employed in all conceivable concentrations and in every possible combination with one another.

The selective agents are preferably employed in a concentration of from 0.005% (w/v) to 1% (w/v) (paraquat, hydrogen peroxide), 5 mM to 500 mM (linolenic acid), 0.001 mM to 1 mM (antimycin A, piericidin A, menadione).

The selective agents are particularly preferably employed in a concentration of from 0.01% (w/v) to 0.3% (w/v) (paraquat), 20 mM to 300 mM (linolenic acid), 0.01 mM to 0.5 mM (antimycin A, piericidin A, menadione).

Preferred combinations of selective agents in the preferred concentration ranges indicated above are hydrogen peroxide+paraquat, hydrogen peroxide+linolenic acid, paraquat+linolenic acid, paraquat+linolenic acid+piericidin A, paraquat+antimycin A, and paraquat+piericidin.

Particular preference is given, in the particularly preferred concentration ranges indicated above, to combinations of hydrogen peroxide and paraquat, of hydrogen peroxide and linolenic acid, of paraquat and linolenic acid, and of paraquat and linolenic acid and piericidin A.

Mutants of S. ruineniae with improved Q10 production are isolated by the following process of the invention. S. ruineniae is mutagenized in the manner described above. Mutants which grow under the selective conditions of the invention are picked and cultivated further. Comparative tests to select mutants with improved Q10 production are carried out by culturing in shake flasks.

Isolated mutants undergo submerged cultivation in a growth medium to generate biomass. The biomass is isolated and Q10 is extracted therefrom in a manner known per se. The amount of Q10 is determined quantitatively by high pressure liquid chromatography (HPLC), but any quantitative method for determining Q10 is suitable.

The mutant with the highest Q10 production is selected from the comparison of Q10 production by the various mutants with the starting strain, and enters a new round of the mutagenesis and selection mentioned. The conditions for the new mutagenesis and selection are selected in this case according to the preferred conditions defined above. It is possible with this method to isolate S. ruineniae strains of the invention which are suitable for biotechnological production of Q10 and are distinctly improved by comparison with the prior art. Such strains are preferably obtained by 1 to 10 rounds of mutagenesis and selection, particularly preferably by 2 to 6 rounds of mutagenesis and selection.

The process of the invention thus makes it possible to obtain distinctly improved Q10-producing strains of S. ruineniae. Distinctly improved Q10-producing strains of S. ruineniae are defined as those whose Q10 productivity is increased by at least 50% compared with the prior art, disclosed in U.S. Pat. No. 4,070,244, of 0.92 mg of Q10/g of biomass. These strains thus have a Q10 productivity of more than 1.38 mg of Q10/g of biomass. The invention thus likewise relates to an S. ruineniae strain which has a Q10 productivity of more than 1.38 mg of Q10/g of biomass and which is obtainable by the mutagenesis and selection process of the invention.

The strain used for the strain improvement by the described process is Sporidiobolus ruineniae Sr-1 (deposited in accordance with the Budapest Treaty under the number DSM 15553 at the Deutsche Sammlung fur Mikroorganismen und Zellkulturen GmbH, D-38142 Brunswick).

A further object of the present invention was to provide an improved process of Q10 production. This object is achieved by a process wherein cells of the Q10-producing strain of S. ruineniae improved according to the invention are cultivated in a culture medium, and the cells are isolated after a growth time of from 70 to 150 h. For Q10 production, the selected strains are cultivated in a culture medium to generate biomass. The culturing takes place in a manner familiar to a person skilled in the art. The culturing for this purpose can take place in shake flasks (laboratory scale) or else by fermentation.

Culture media familiar to the skilled worker are described in the literature (U.S. Pat. No. 4,070,244) and typically consist of a carbon source (C source), a nitrogen source (N source) and additions such as vitamins, salts and trace elements, by which cell growth is optimized. C sources are those which can be used by S. ruineniae to form biomass. These include glucose, dextrose, maltose, acetate (acetic acid), glycerol and ethanol. C sources utilizable in the culturing of strain of the genus Sporidiobolus are known in the prior art. A survey is given for example by the Internet address (www.cbs.knaw.nl/cbshome.html) of the CBS, “Centraalbureau voor Schimmelcultures”, the Netherlands' collection of strains.

Preferred C sources for culturing strains of the genus Sporidiobolus are glucose, starch, maltodextrin, glycerol, maltose, acetic acid and salts thereof, sucrose, molasses, citric acid and salts thereof.

N sources are those which can be utilized by Sporidiobolus ruineniae to form biomass. These include ammonia, gaseous or in aqueous solution as NH₄OH, or else its salts such as, for example, ammonium sulfate, ammonium chloride or ammonium nitrate. Also suitable as N source are the known nitrate salts such as, for example, KNO₃, NaNO₃, ammonium nitrate, Ca(NO₃)₂, Mg(NO₃)₂, and other N sources such as urea. The N sources also include complex amino acid mixtures such as yeast extract (including technical yeast extracts such as, for example, HY Yest444 from Quest), proteose peptone, malt extract, soybean peptone, casamino acids, corn steep liquor, and NZ amines and yeast nitrogen base.

Culturing can take place in the so-called batch mode, in which case the culture medium is inoculated with a starter culture of Sporidiobolus ruineniae and then cell growth takes place without further feeding with nutrient sources.

Culturing can also take place in the so-called fed-batch mode, in which case an initial growth phase in batch mode is followed by additional feeding with nutrient sources (feed) in order to compensate for consumption thereof. The feed can consist of the C source, the N source and of a mixture of the two. In addition it is also possible to add other media constituents and addition which specifically increase Q10 production to the feed.

Culturing in fed-batch mode is preferred.

Preferred C sources in the feed are glycerol, glucose, sucrose, molasses, maltose or acetate.

Preferred N sources in the feed are ammonia, gaseous or in aqueous solution as NH₄OH and its salts ammonium sulfate and ammonium chloride, also KNO₃, NaNO₃ and ammonium nitrate, yeast extract, HY Yest444, proteose peptone, malt extract, soybean peptone, casamino acids, corn steep liquor, and NZ amines and yeast nitrogen base.

Particularly preferred N sources in the feed are ammonia, or ammonium salts, yeast extract, soybean peptone, malt extract or corn steep liquor.

It is also possible to add to the culture medium components which are specifically suitable for increasing Q10 production (so-called inducers). These include compounds such as p-hydroxybenzoic acid and its salts and esters (U.S. Pat. No. 4,070,244), isopentenol (U.S. Pat. No. 4,220,719), tyrosine and its biochemical precursors such as shikimic acid or chorismic acid and unsaturated fatty acids such as oleic acid (R. A. Hagerman et al., Free Radical Research (2002) 36: 485-490).

Culturing takes place under conditions of pH and temperature which favor the growth and Q10 production of Sporidiobolus ruineniae. The useful pH range extends from pH 4 to pH 10. A pH range from pH 4 to pH 9 is preferred. A pH range from pH 5.0 to pH 8.5 is particularly preferred.

The preferred temperature range for growth of S. ruineniae is 20° C. to 40° C. The temperature range from 25° C. to 35° C. is particularly preferred.

A sufficient supply of oxygen is necessary for growth of S. ruineniae. The oxygen supply is preferably ensured by introducing compressed air or pure-oxygen.

The duration of growth of S. ruineniae for Q10 production is between 10 h and 200 h. A growth time of from 30 h to 120 h is preferred. A growth time of from 40 h to 100 h is particularly preferred.

S. ruineniae cells obtained by the process described above for culturing contain the Q10 product. The Q10 can be extracted from these cells and isolated. The cells can, however, also be processed directly for further use without isolating the Q10 therefrom.

Methods for isolating Q10 are known (U.S. Pat. No. 4,070,244) and comprise an extraction step in which Q10 must be detached from the cell membrane. This step comprises destruction of the cell structure, which can take place either mechanically (by grinding) or chemically with solvents. The Q10 is then extracted into an organic solvent and purified from contaminating constituents. This can take place by precipitation methods, chromatographic processes or else by specific complexation (e.g. cyclodextrins). Various analytical methods for identifying the isolated Q10 are available, including, inter alia, mass spectroscopy, NMR, UV/Vis absorption spectroscopy, HPLC or else a combination thereof.

The preferred analytical method for Q10 is HPLC.

Methods for processing the Sporidiobolus ruineniae cells from the culture for direct further use without previous purification of the Q10 contained therein are freeze-drying, spray drying, thermal drying, and grinding and sieving of the dried biomass obtained. Under suitable conditions, the Q10 processed in this way can be directly used further for an application.

### DETAILED DESCRIPTION OF PREFERRED EMBODIMENTS

Other objects and features of the present invention will become apparent from the following detailed description considered in connection with the accompanying examples which disclose several embodiments of the present invention. It should be understood, however, that the examples are designed for the purpose of illustration only and not as a definition of the limits of the invention.

is 20° C. to 40° C. The temperature range from 25° C. to 35° C. is particularly preferred.

A sufficient supply of oxygen is necessary for growth of S. ruineniae. The oxygen supply is preferably ensured by introducing compressed air or pure oxygen.

The duration of growth of S. ruineniae for Q10 production is between 10 h and 200 h. A growth time of from 30 h to 120 h is preferred. A growth time of from 40 h to 100 h is particularly preferred.

S. ruineniae cells obtained by the process described above for culturing contain the Q10 product. The Q10 can be extracted from these cells and isolated. The cells can, however, also be processed directly for further use without isolating the Q10 therefrom.

Methods for isolating Q10 are known (U.S. Pat. No. 4,070,244) and comprise an extraction step in which Q10 must be detached from the cell membrane. This step comprises destruction of the cell structure, which can take place

either mechanically (by grinding) or chemically with solvents. The Q10 is then extracted into an organic solvent and purified from contaminating constituents. This can take place by precipitation methods, chromatographic processes or else by specific complexation (e.g. cyclodextrins). Various analytical methods for identifying the isolated Q10 are available, including, inter alia, mass spectroscopy, NMR, UV/Vis absorption spectroscopy, HPLC or else a combination thereof.

The preferred analytical method for Q10 is HPLC.

Methods for processing the Sporidiobolus ruineniae cells from the culture for direct further use without previous purification of the Q10 contained therein are freeze-drying, spray drying, thermal drying, and grinding and sieving of the dried biomass obtained. Under suitable conditions, the Q10 processed in this way can be directly used further for an application.

### DETAILED DESCRIPTION OF PREFERRED EMBODIMENTS

Other objects and features of the present invention will

become apparent from the following detailed description considered in connection with the accompanying examples which disclose several embodiments of the present invention. It should be understood, however, that the examples are designed for the purpose of illustration only and not as a definition of the limits of the invention.

The following examples serve to explain the invention further.

### EXAMPLE 1

### Isolation of Q10

Q10 Isolation Without Mechanical S. ruineniae Cell Disruption:

The method was employed in the screening of mutants with improved Q10 production. 50 ml of YPGC medium (1% yeast extract, 2% peptone, 5% glycerol, 2% corn steep liquor) were inoculated with Sporodiobolus ruineniae Sr-1 (DSM 15553) and incubated at 28° C. on an orbital shaker (Infors, 140 rpm) for 120 h. The batch was transferred into a 50 ml centrifuge tube (Falcon tube), and the S. ruineniae cells were spun down (Heraeus Megafuge 1.0R, 3000 rpm for 10 min), washed in 30 ml of deionized water, resuspended in 5 ml of deionized water and transferred into a 50 ml round-bottom flask. The cell suspension was then frozen as uniformly as possible in an ethanol/dry ice mixture and lyophilized (Christ “alpha 2-4” freeze drier). 200 mg of dry matter were weighed into a 12 ml glass tube, mixed with 10 ml of ethanol and blanketed with gaseous nitrogen. Extraction was carried out by horizontal shaking at 40° C. overnight. The suspension was then centrifuged, and the supernatant was collected and blanketed with nitrogen. In a second extraction, the precipitate was taken up in 8-10 ml of ethanol, blanketed with nitrogen, thoroughly mixed and shaken horizontally at 40° C. for 2 hours. In a third extraction, the procedure was repeated and the suspension was shaken horizontally at 40° C. for 1 hour. The supernatants from extractions 1-3 were combined, mixed with 5 ml of deionized water and 10 ml of n-heptane and centrifuged to separate the phases. The upper, colored n-heptane phase was pipetted into a 50 ml round-bottom flask, and the lower phase was again extracted by shaking with 10 ml of n-heptane and centrifuged to separate the phases. The n-heptane phases were combined and the solvent was removed in a rotary evaporator. A reddish residue remained and was taken up in 1 ml of THF and used for the quantitative determination by calibrated HPLC.

Q10 Isolation with Mechanical S. ruineniae Cell Disruption:

The method was employed for Q10 determination in samples from a fermenter. 25 ml of cell suspension taken from a fermentation (see 6th and 7th example) was transferred into a 50 ml centrifuge tube (Falcon tube), the S. ruineniae cells were spun down (Heraeus Megafuge 1.0R, 3000 rpm for 10 min) and the cell pellet was washed once with water. The cell pellet was suspended in 10 ml of water and quantitatively transferred into a weighed 50 ml round-bottom glass flask. The cell suspension was frozen, freeze dried overnight (Christ “alpha 2-4” freeze drier) and then determined by weighing the dried biomass. This was followed by mechanical cell disruption using a Dismembrator (Braun, Melsungen). For this purpose, 300 mg of the dried biomass were weighed into a 3 ml Teflon chamber suitable for the Dismembrator, a steel ball (diameter 5 mm) was added, and the Teflon chamber was closed and frozen in liquid nitrogen. The deep-frozen Teflon chamber was then clamped in the Dismembrator and shaken at 2000 excursions/min for 2 min. 200 mg of the dry biomass mechanically disrupted in this way were weighed into a 12 ml glass tube, mixed with 8 ml of n-heptane and shaken at 40° C. overnight. The batch was then centrifuged (Heraeus centrifuge, 3000 rpm for 10 min), and the organic phase was transferred into a 100 ml round-bottom glass flask. Extraction with n-heptane was then repeated twice (shaking at 40° C. for 2 h each time). The n-heptane phases were combined and the solvent was removed in a rotary evaporator. A reddish residue remained and was taken up in 1 ml of THF and used for the quantitative determination by calibrated HPLC.

Quantification of Q10 Production by Calibrated HPLC:

Calibrated HPLC was used for quantification of Q10-containing samples. An Agilent 1100 HPLC equipped with an Elite Hy-Purity C18 column (manufactured by Phenomenex) was used. The mobile phase (isocratic) was composed of 71% (v/v) acetonitrile, 22% (v/v) dichloromethane, 4% (v/v) methanol, 2% H₂O, and 1% (v/v) propionic acid. The flow rate was 2 ml/min. The column temperature was room temperature. The volume loaded was 5 μl. The detector was an HP diode array detector (Hewlett Packard) combined with an integrator to quantify the peak area. Measurement took place at a wavelength of 280 nm. A 0.2 mg/ml standard solution of Q10 (Sigma) in THF was used for calibration. The retention time of Q10 under the stated conditions was 10.3 min. The UV spectrum of the peak served to identify and determine the purity of the product peaks in samples for quantification. The amount of Q10 was determined quantitatively by comparison of the peak area for the Q10 standard solution with the Q10 peak area for unknown samples. The volumetric Q10 production (mg of Q10/l of culture medium) and the Q10 productivity (mg of Q10/g of dry biomass) was determined from the quantitative HPLC determination.

### EXAMPLE 2

### Mutagenesis of Sporidiobolus ruineniae with UV Radiation or N-methyl-N′-nitro-N-nitrosoguanidine (NG)

UV Mutagenesis On YPD Plates:

Sporidiobolus ruineniae Sr-1 was cultivated in a shake flask culture in YPD medium (1% yeast extract, 2% peptone, 2% glucose) at 28° C. for 48 h. The culture was then diluted to an optical density OD600 of 0.1, and 0.1 ml of this was streaked on YPD agar plates (1% yeast extract, 2% peptone, 2% glucose, 1.5% agar). The plate was then exposed to UV light (UV light source: TFP 20 M UV screen, 6×15 W, 213 nm from Vilber Lourmat, France). The radiation dose (exposure time and intensity) which led to the desired kill rate of Sporidiobolus ruineniae cells was determined in preliminary tests. The kill rate should be between 50% and 99%. Cells UV-mutagenized in this way were incubated at 28° C. for 2-5 days, until colonies were visible. YPD plates with additions which exerted a selection pressure on Sporidiobolus ruineniae and created a growth advantage for Q10-overproducing mutants were used in parallel batches (see Examples 3 and 4).

UV Mutagenesis in Cell Suspension

Sporidiobolus ruineniae Sr-1 was cultivated in a shake flask culture in YPD medium (1% yeast extract, 2% peptone, 2% glucose) at 28° C. for 48 h. Fresh YPD medium was inoculated with the preculture and further cultivated under the same conditions until the S. ruineniae cells reached an OD₆₀₀ of 0.4. 0.5 ml of this cell suspension was spun down, resuspended in 2 ml of 0.9% NaCl solution and exposed to a UV treatment in a sterile Petri dish for 20, 30 and 40 seconds (UV light source: TFP 20 M UV screen, 6×15 W, 213 nm from Vilber Lourmat, France). The mutagenized cells were then put on YPD plates and incubated at 28° C. for 2-5 days, until colonies were visible. YPD plates with additions which exerted a selection pressure on Sporidiobolus ruineniae and created a growth advantage for Q10-overproducing mutants were used in parallel batches (see Examples 3 and 4).

NG Mutagenesis

Sporidiobolus ruineniae Sr-1 was cultivated in a shake flask culture in YPD medium (1% yeast extract, 2% peptone, 2% glucose) at 28° C. for 48 h. Fresh YPD medium was inoculated with this preculture and cultivated further under the same conditions until the S. ruineniae cells reached an OD₆₀₀ of 1.0. 10 ml of this cell suspension were spun down, washed in 10 ml of citrate buffer (0.1 M Na citrate, pH 5.5) and incubated in 1 ml portions in citrate buffer with various amounts of NG (0.01 to 0.06 mg/ml) in a test tube at 28° C. for one hour. The cells were then spun down, washed in 1 ml of phosphate buffer (0.1 M KH₂PO₄, pH 7.0 adjusted with NaOH) and put in various dilutions on YPD plates. The plates were incubated at 28° C. for 2-5 days, until colonies were visible. YPD plates with additions which exerted a selection pressure on Sporidiobolus ruineniae and created a growth advantage for Q10-overproducing mutants were used in parallel batches (see Examples 3 and 4).

The methods described herein are suitable for isolating mutants of Sporidiobolus ruineniae with improved Q10 production. This process is particularly suitable for improving Q10 production by S. ruineniae to a marked extent by repeated rounds of mutagenesis and selection in which a mutant with improved Q10 production is selected and used as starting strain for the next round of mutagenesis and selection (see following Examples).

### EXAMPLE 3

### Selection of Sporidiobolus ruineniae Mutants with Improved Q10 Production

Mutants of S. ruineniae were produced as described in Example 2 and cultivated under selective conditions. The selective growth conditions were chosen so that preferential isolation was possible of those mutants which ought to be capable of increased Q10 production owing to a mutation in Q10 metabolism.

Selective agents were employed in concentrations which reduced the growth of the starting strain used for the mutagenesis by 50-90%. The selective agents were, unless described otherwise, admixed to the YPD plates on which the mutagenized cells produced as in Example 2 were plated out.

Specifically, the following selective conditions were chosen: Paraquat was added in concentrations of 0.1-0.5% (w/v) to the YPD plates. Menadione (2-methyl-1,4-naphthoquinone) was added in a concentration of 3 μg/ml, 20 μg/ml, 50 μg/ml or 100 μg/ml to the YPD plates. Antimycin was admixed to the YPD plates in concentrations between 5 and 100 μM, piericidin A in a concentration of 20, 50 or 100 μM.

### EXAMPLE 4

### Selection of Sporidiobolus ruineniae Mutants by Combinations of Various Additions

Mutagenized Sporidiobolus ruineniae cells with improved Q10 production were also isolated by a combination of various selective agents.

Selection with a Combination of Paraquat and Linolenic Acid:

S. ruineniae Sr-1 cells were mutagenized by NG treatment as described in Example 2 and then incubated further in a shake culture under selective conditions at 2° C. for 4 h. YPD medium was employed for the shake culture with addition of a combination of the selective agents paraquat, in a concentration of 0.01% and linolenic acid, in a concentration of 50 mM or 100 mM. After incubation at 28° C. for 4 h, the cells were plated out on YPD plates.

Selection with a Combination of Paraquat, Linolenic Acid and Piericidin A:

S. ruineniae Sr-1 cells were mutagenized by NG treatment as described in Example 2 and then incubated further in a shake culture under selective conditions at 28° C. for 4 h. YPD medium was employed for the shake culture with addition of a combination of the selective agents paraquat, in a concentration of 0.01%, and linolenic acid, in a concentration of 50 mM or 100 mM. After incubation at 28° C. for 4 h, the cells were plated out on YPD plates, which additionally contained piericidin A in a concentration of 50 μM.

YPD plates which had been inoculated with the S. ruineniae cells pretreated in the manner described were incubated at 28° C. for 2-5 d. Single colonies of the S. ruineniae mutants produced in this way were picked and plated out on fresh YPD plates and again incubated. 400 mutants were isolated in this way in each mutagenesis round and were then investigated for increased Q10 production (Example 5).

### EXAMPLE 5

### Analysis of Q10 Production by Sporidiobolus ruineniae Mutants

Mutants of Sporidiobolus ruineniae were isolated by the mutagenesis processes described in Example 2 and the selection process described in Examples 3 and 4. Two rounds of screening were carried out. 400 mutants were picked in each round of screening and were cultivated on YPD plates for the subsequent Q10 analysis. The Q10 analysis took place by culturing the mutants in a shake flask and subsequent determination of Q10 production.

50 ml of YPGC medium (1% yeast extract, 2% peptone, 5% glycerol, 2% corn steep liquor) were in each case inoculated with one mutant and incubated at 28° C. and a shaker speed of 140 rpm for 120 h. The cells were then spun down, washed in 30 ml of deionized water, resuspended in 5 ml of deionized water and transferred into a weighed 50 ml round-bottom flask. The cell suspension was then frozen as uniformly as possible in an ethanol/dry ice mixture and lyophilized. The weight of lyophilized biomass was determined as dry matter. 200 mg of the dry matter were weighed out, mixed with 10 ml of ethanol and blanketed with gaseous nitrogen. Q10 extraction took place as described in Example 1 (Q10 isolation without mechanical disruption). The volumetric Q10 production (mg Q10/l) and the specific Q10 production (mg of Q10/g of biomass) were determined. The starting strain used for the mutagenesis was used as reference in each screening series. The strain with the most improved Q10 volumetric production was selected from in each case 400 mutants by repeat experiments and was employed as starting strain for the next screening round.

Table 1 shows the Q10 production by a selection of mutants from the first screening round, these having been produced as described in Examples 1, 2 and 3 and being distinguished by distinctly improved Q10 production compared with the starting strain. Mutagenesis of S. ruineniae in the first screening round took place by UV mutagenesis.

TABLE 1
Sporidiobolus ruineniae Sr-1 (DSM 15553) and mutants with
improved Q10 production.

| | | Q10 production | Q10 production |
|---|---|---|---|
| Strain | Selection | (mg/g) | (mg/l) |
| Sr-1 | — | 0.95 | 8.2 |
| Sr-5 | 0.1% paraquat | 1.45 | 10.8 |
| Sr-15 | 0.1% paraquat | 2.20 | 13.5 |
| Sr-43 | 0.1 mM menadione | 1.51 | 15.7 |
| Sr-61 | 0.1 mM menadione | 1.81 | 13.2 |
| Sr-176 | 0.1 mM antimycin | 1.66 | 10.3 |
| Sr-178 | 0.1 mM antimycin | 1.59 | 11.0 |
| Sr-209 | 0.01% linolenic acid | 1.66 | 13.6 |
| Sr-220 | 0.01% linolenic acid | 2.10 | 17.8 |

Strain Sr-220 was selected from the first screening round for the second screening round. In the second screening round, mutants of the strain Sr-220 were generated by NG mutagenesis. The mutants were selected by a combination of paraquat and linolenic acid. A selection of mutants with greatly improved Q10 production is listed in Table 2.

TABLE 2
Sporidiobolus ruineniae strain Sr220 and mutants with
improved Q10 production.

| | | Q10 production | Q10 production |
|---|---|---|---|
| Strain | Selection | (mg/g) | (mg/l) |
| Sr220 | — | 2.18 | 18.1 |
| Sr220-87 | 0.1% paraquat + | 2.34 | 19.3 |
| | 0.01% linolenic acid | | |
| Sr220-121 | 0.1% paraquat + | 3.50 | 22.9 |
| | 0.01% linolenic acid | | |
| Sr220-148 | 0.1% paraquat + | 2.66 | 20.9 |
| | 0.01% linolenic acid | | |
| Sr220-159 | 0.1% paraquat + | 2.94 | 24.8 |
| | 0.01% linolenic acid | | |
| Sr220-218 | 0.1% paraquat + | 2.29 | 19.7 |
| | 0.01% linolenic acid | | |
| Sr220-304 | 0.1% paraquat + | 2.71 | 21.8 |
| | 0.01% linolenic acid | | |
| Sr220-307 | 0.1% paraquat + | 2.57 | 20.2 |
| | 0.01% linolenic acid | | |

The strain Sr220-159 was selected from the second screening round in order to develop a process for fermentation of S. ruineniae (see Example 6 and Example 7). Comparison of the S. ruineniae wild-type strain Sr-1 and of the mutant Sr220-159 shows that Q10 production by S. ruineniae can be improved significantly by the novel strain-improvement process. The Q10 productivity (mg of Q10/g of biomass) and the volumetric production (mg of Q10/l of culture medium) was in each case increased by about 300% by two rounds of mutagenesis and selection.

### EXAMPLE 6

### Batch Fermentation of S. ruineniae

A Biostat M fermenter (Braun, Melsungen) was used for the batch fermentation. The culture vessel had a total volume of 2 l. The working volume was 1 l. The fermenter was additionally equipped with electrodes to measure the pO₂ and the pH. The pH of the fermentation was 6.0 and was controlled with ammonium hydroxide and phosphoric acid. Compressed air was passed into the fermentation vessel at 1.6 l/min. The fermentation temperature was 28° C., and the stirring speed was 900 rpm. The fermenter was additionally equipped with a foam sensor which controlled the metering in of Structol J 647 antifoam (Schill und Seilacher), diluted 1:5 with H₂O. The fermenter was charged with 1 l of sterile YPGC medium (Example 1). To generate an inoculum, a first preculture of the strain Sporidiobolus ruineniae Sr220-159 was produced in the same medium at 28° C., 140 rpm (Infors shaker) for four days. 5 ml of the first preculture were used to inoculate a second preculture in 50 ml of YPGC medium. The second preculture was shaken at 28° C. and 140 rpm overnight. The second preculture was used to inoculate the fermenter under sterile conditions, and the fermentation was started. The fermentation time was 96 h. After completion of the fermentation, the dry biomass and Q10 were determined as described in the 1st example. The dried biomass amounted to 25 g/l and the specific Q10 yield amounted to 0.89 mg of Q10/g of dry biomass.

### EXAMPLE 7

### Fed Batch Fermentation of S. ruineniae

A Biostat M fermenter (Braun, Melsungen) was used for the fed batch fermentation. The culture vessel had a total volume of 2 l. The working volume was 1 l. The fermenter was additionally equipped with electrodes to measure the pO₂ and the pH. The pH of the fermentation was 6.0 and was controlled with ammonium hydroxide and phosphoric acid. Compressed air was passed into the fermentation vessel at 1.6 l/min. The fermentation temperature was 28° C., and the stirring speed was 900 rpm. The fermenter was additionally equipped with a foam sensor which controlled the metering in of Structol J 647 antifoam (Schill und Seilacher), diluted 1:5 with H₂O. The fermenter was charged with 1 l of sterile YPGC medium (Example 1). To generate an inoculum, a first preculture of the strain Sporidiobolus ruineniae Sr220-159 was produced in the same medium at 28° C., 140 rpm (Infors shaker) for four days. 5 ml of the first preculture were used to inoculate a second preculture in 50 ml of YPGC medium. The second preculture was shaken at 28° C. and 140 rpm overnight. The second preculture was used to inoculate the fermenter under sterile conditions, and the fermentation was started. The fermentation time was 96 h. The feeding was started 24 h after the start of the fermentation. The feeding solution consisted of 75% (v/v) glycerol, 2% (w/v) yeast extract. The feeding rate was 12 ml/h from 24 h to 48 h of the fermentation, and 6 ml/h from 48 h to the end of the fermentation (96 h). At various times, fermenter medium was obtained for quantitative determination of the Q10 yield. The Q10 determination took place by the Dismembrator method (see Example 1). At the end of the fermentation the biomass amounted to 73.6 g/l and the Q10 yield amounted to 3.12 mg/g of dry biomass. This corresponds to a significant increase compared with batch fermentations and the prior art for Sporidiobolus ruineniae (U.S. Pat. No. 4,070,244, Q10 production 0.92 mg/g, Biomasse 18 g/l).

Accordingly, while a few embodiments of the present invention have been shown and described, it is to be understood that many changes and modifications may be made thereunto without departing from the spirit and scope of the invention as defined in the appended claims.

What is claimed is:

1. A process for producing a mutagenized Sporidiobolus ruineniae strain having a Q10 productivity of greater than 1.38 mg of Q10/g of dry biomass, comprising
(a) subjecting a Sporidiobolus ruineniae strain to genetic manipulation by mutagenesis;
(b) subjecting mutagenized Sporidiobolus ruineniae strain to a selection where the mutagenized Sporidiobolus ruineniae strain is cultivated under conditions which inhibit growth of the Sporidiobolus ruineniae strain employed in (a), the conditions being chosen so that the mutagenized strain overcomes the growth inhibition through a Q10 production which is increased by comparison with the Sporidiobolus ruineniae strain employed in (a), grows in a fermentation medium; and
(c) isolating the mutagenized strain from the fermentation medium.

2. The process as claimed in claim 1,
wherein the mutagenesis is selected from the group consisting of an action of mutagenic substances, and high-energy radiation on the S. ruineniae genome, and the kill rate in the mutagenesis is from 30 to 99%.

3. The process as claimed in claim 1,
wherein selection of the Sporidiobolus ruineniae strain with a Q10 productivity of greater than 1.38 mg of Q10/g of dry biomass is brought about by conditions which generate an oxidative stress.

4. The process as claimed in claim 3, comprising
generating the oxidative stress by a substance selected from the group consisting of paraquat, hydrogen peroxide unsaturated fatty acids, and linolenic acid.

5. The process as claimed in claim 1,
wherein selection of the Sporidiobolus ruineniae strain with a Q10 productivity of greater than 1.38 mg of Q10/g of dry biomass is effected by an inhibitor of the respiratory chain.

6. The process as claimed in claim 5,
wherein the inhibitor of the respiratory chain is selected from the group consisting of antimycin A, piericidin, mucidin, rotenone, and menadione.

7. The process as claimed in claim 1,
wherein selection of the Sporidiobolus ruineniae strain with a Q10 productivity of greater than 1.38 mg of Q10/g of dry biomass takes place through inhibition of a step in the Q10 biosynthetic pathway of Sporidiobolus ruineniae.

8. The process as claimed in claim 7,
wherein the inhibition of a step of the Q10 biosynthetic pathway of Sporidiobolus ruineniae is effected by a substance selected from the group consisting of glyphosate, lovastatin, cerivastatin, atorvastatin, compactin, or ethionine.

9. A mutated Sporidiobolus ruineniae strain obtainable by means of a process as claimed in claim 1, which strain has a Q10 productivity of more than 1.38 mg of Q10/g of biomass.

10. A process for the production of Q10, comprising
cultivating cells of a strain as claimed in claim 9 in a culture medium;
isolating the cells after a growth period; and
isolating Q10 therefrom.

11. The process as claimed in claim 10,
wherein the cells are cultured in fed-batch mode, where an initial growth phase in batch mode is followed by additional feeding of nutrient sources in a feed in order to compensate for consumption thereof.

12. The process as claimed in claim 11,
wherein the nutrient sources in the feed are selected from the group consisting of a C source, an N source and mixture thereof.

13. The process as claimed in claim 12,
wherein the C source in the feed is selected from the group consisting of glycerol, glucose, sucrose, molasses, maltose, and acetate and the N source is selected from the group consisting of ammonia, ammonia salts, yeast extract, soybean peptone, malt extract, and corn steep liquor.
